(19) United States
(12) Patent Application Publication
(10) Pub. No.: US 2026/0294825 A1
(43) Pub. Date: Oct. 1, 2026

(54) DISK-BASED MICROFLUIDIC PLATFORM FOR MANUFACTURING LIPID NANOPARTICLES USING CENTRIFUGAL FORCE AND METHOD USING SAME

(71) Applicant: Korea University Research and Business Foundation, Seoul (KR)

(72) Inventors: ARAM CHUNG, Seoul (KR); Chae Lim AN, Incheon (KR); Dong Hwan SEO, Seoul (KR)

(73) Assignee: Korea University Research and Business Foundation, Seoul (KR)

(21) Appl. No.: 19/471,194

(22) PCT Filed: Nov. 14, 2024
(86) PCT No.: PCT/KR2024/017930

§ 371 Date: Sep. 30, 2025

(30) Foreign Application Priority Data
Nov. 14, 2023 (KR) 10-2023-0157325
Nov. 13, 2024 (KR) 10-2024-0160885

(51) Int. Cl. A61K 9/51; B01J 13/08

(52) U.S. Cl. CPC A61K 9/5123; A61K 9/5192; B01J 13/08

(57) ABSTRACT

The present invention relates to a microfluidic platform for producing lipid nanoparticles (LNPs), which comprises a microfluidic channel for producing lipid nanoparticles inside using centrifugal force, at least one inlet configured to supply a fluid into the microfluidic channel, and at least one outlet configured to discharge the fluid from the microfluidic channel, the platform being provided in the form of a disk member.

### TECHNICAL FIELD

The present invention relates to a centrifugal disk-based microfluidic platform for producing lipid nanoparticles and a method of using the same. More particularly, the present invention pertains to a centrifugal disk-based microfluidic platform and a method of using the same, which are capable of producing lipid nanoparticles with high efficiency.

### BACKGROUND ART

Lipid nanoparticles (LNPs) can be used as carriers for transporting biologically active substances such as small-molecule drugs, proteins, and nucleic acids into cells and/or intracellular compartments. LNPs are typically produced by physically mixing their major components. Depending on the mixing method employed, the size, physical properties, and encapsulation efficiency of the lipid nanoparticles may vary significantly. Methods conventionally used to produce lipid nanoparticles include pipette mixing and microfluidic mixing. Pipette mixing is a simple method in which two types of solutions are mixed using a pipette. This approach has the advantages of accessibility and low cost, and it is mainly employed in studies using small volumes. However, pipette mixing has disadvantages in that the size of the lipid nanoparticles cannot be easily controlled, and the encapsulation efficiency of the resulting lipid nanoparticles is low.

Microfluidic mixing, on the other hand, is carried out by connecting a mixing chip, in which two types of solutions are mixed, to a syringe pump, or by employing a mixing system such as a NanoAssembler. Microfluidic mixing has advantages in that lipid nanoparticles can be reproducibly produced with uniformity, the particle size can be controlled at a small scale, and relatively high encapsulation efficiency can be achieved. However, microfluidic mixing also suffers from drawbacks such as a large dead volume occurring inside devices like mixing chips, and difficulty in controlling production volume, making it unsuitable for either large-scale production or extremely small-scale preparation. In addition, since a specialized mixing system must be used, the associated cost is relatively high.

Accordingly, with the increasing utilization of lipid nanoparticles in various applications, there has been growing research directed toward producing LNPs with consistent physical properties and particle sizes across batches, while enabling facile large-scale production.

### SUMMARY OF THE INVENTION

### Technical Object

It is an object of the present invention to provide a centrifugal disk-based microfluidic platform and a method of using the same for producing lipid nanoparticles having a controlled size with high production efficiency.

Another object of the present invention is to provide a centrifugal disk-based microfluidic platform and a method of using the same, which are capable of mass-producing lipid nanoparticles of uniform size for various applications.

### Technical Solutions

According to one aspect of the present invention, embodiments of the invention include a microfluidic platform for producing lipid nanoparticles and a method using the microfluidic platform.

In an embodiment, the microfluidic platform for producing lipid nanoparticles comprises a microfluidic channel utilizing centrifugal force and producing lipid nanoparticles (LNPs) therein; one or more inlets supplying fluid to the microfluidic channel; one or more outlets through which the fluid is discharged from the microfluidic channel; and a disk member rotating the fluid.

In an embodiment, the microfluidic channel extends in one direction, the inlet and the outlet are respectively connected to one end and the other end of the microfluidic channel, and extend upward relative to the microfluidic channel. The inlet is provided at a first angle with respect to a first plane parallel to the ground, and the outlet is provided at a second angle with respect to the first plane.

The first angle is 20° to 85°, and the second angle is not more than 85°. As the first angle and the second angle decrease, the size of the lipid nanoparticles decreases.

The first angle and the second angle may be the same and within the range of 45° to 70°.

The inner diameter of the microfluidic channel is 100 μm to 300 μm, and the Reynolds number of the fluid in the microfluidic channel is 5 to 1520.

The disk member rotates to generate centrifugal force inside the microfluidic channel.

The rotational speed of the disk member is 500 rpm to 4000 rpm, the average diameter of the lipid nanoparticles is 10 nm to 300 nm, and the polydispersity index (PDI) is 0.20 or less.

The inlet comprises two or more inlets through which a lipid mixture and an active substance mixture flow into the microfluidic channel, and the average velocity of the fluid at the inlet is 1 μL/s to 200 μL/s.

The lipid mixture comprises ionizable lipids, helper lipids, stabilizing lipids, and PEG-lipids. The active substance mixture comprises one or more selected from the group consisting of chemotherapeutic agents, small-molecule drugs, proteins, and nucleic acids.

The N/P ratio of the lipid mixture to the active substance mixture is 1 to 30.

The microfluidic platform further comprises one or more microfluidic chips including the microfluidic channel, the one or more inlets, and the one or more outlets, and mounted on one surface of the disk member. The microfluidic chip is detachably mounted to the disk member in a reversible manner.

The microfluidic chip comprises a body portion in which the microfluidic channel, the inlet, and the outlet are formed; and one or more cover portions covering one or both of the first and second surfaces of the body portion. The microfluidic channel is concavely formed inward from one surface of the body portion, and the inlet and the outlet are formed to penetrate the body portion.

The cover portion comprises a first cover and a second cover. The first cover covers one surface of the body portion and includes one or more holes corresponding to the inlet and the outlet, and the second cover covers the other surface of the body portion.

One surface of the disk member further comprises one or more seating portions on which the microfluidic chip is mounted. The seating portion corresponds in size to the microfluidic chip, is concavely formed inward from the surface of the disk member, and receives the lower surface of the microfluidic chip.

The microfluidic chip has a box shape and is arranged radially with respect to the center of the disk member. The inlet is disposed adjacent to the center of the disk member, and the outlet is disposed adjacent to the rim of the disk member.

The microfluidic channel comprises at least one of a herringbone mixer pattern, a serpentine mixer pattern, and a toroidal mixer pattern.

The method of using the microfluidic platform for producing lipid nanoparticles comprises preparing a lipid mixture and an active substance mixture; injecting the lipid mixture and the active substance mixture into the inlet; and rotating the disk member.

The inlet comprises two or more inlets through which the lipid mixture and the active substance mixture are introduced into the microfluidic channel. The average velocity of the fluid at the inlet is 1 μL/s to 200 μL/s, and the rotational speed of the disk member is 500 rpm to 4000 rpm.

The inlet is connected to one end of the microfluidic channel, and the Reynolds number of the fluid in the microfluidic channel is 5 to 1520.

The lipid mixture and the active substance mixture are injected into the inlet so that the N/P ratio is 1 to 30, and the lipid mixture and the active substance mixture are mixed in the microfluidic channel by centrifugal force, thereby producing lipid nanoparticles. The lipid nanoparticles are discharged through the outlet connected to the other end of the microfluidic channel, and the average diameter of the lipid nanoparticles is 10 nm to 300 nm.

### Advantageous Effects

As described above, according to the present invention, it is possible to produce lipid nanoparticles of uniform size with high encapsulation efficiency by using a centrifugal disk-based microfluidic platform for producing lipid nanoparticles and a method of using the same.

In addition, according to the present invention, the centrifugal disk-based microfluidic platform and the method of using the same enable facile mass production of lipid nanoparticles and provide lipid nanoparticles suitable for various applications.

### BRIEF DESCRIPTION OF THE DRAWINGS

FIG. 1 is a schematic view of a microfluidic platform for producing lipid nanoparticles according to an embodiment of the present invention.

FIG. 2 illustrates the angles of the inlet and outlet of the microfluidic chip of FIG. 1.

FIG. 3 is a view showing the upper surface of the disk member of FIG. 1.

FIG. 4 illustrates a microfluidic channel according to an embodiment of the present invention.

FIG. 5 is a schematic view of a microfluidic platform for producing lipid nanoparticles according to another embodiment of the present invention.

FIG. 6 is a schematic view of a microfluidic platform for producing lipid nanoparticles according to yet another embodiment of the present invention.

FIG. 7 is a view showing the structure of FIG. 6 manufactured by 3D printing.

FIG. 8 is a schematic view of a microfluidic platform for producing lipid nanoparticles used in an embodiment.

FIG. 9 shows the particle size of lipid nanoparticles according to the inlet angle of the microfluidic chip in an embodiment of the present invention.

FIG. 10 shows a comparison of lipid nanoparticles produced by the microfluidic platform of an embodiment of the present invention and by pipette mixing of a comparative example.

FIG. 11 shows a comparison of lipid nanoparticles produced by the microfluidic platform of an embodiment of the present invention and by syringe mixing of a comparative example.

FIG. 12 shows the intracellular delivery efficiency according to a method of producing lipid nanoparticles containing eGFP-mRNA.

FIG. 13 shows the intracellular delivery efficiency according to a method of producing lipid nanoparticles containing F-Luc-mRNA.

FIG. 14 shows the delivery efficiency to HEK293 cells of lipid nanoparticles produced using the microfluidic platform of the present invention.

FIG. 15 shows the delivery efficiency to CHO-K1 cells of lipid nanoparticles produced using the microfluidic platform of the present invention.

FIG. 16 shows the delivery efficiency to Jurkat cells of lipid nanoparticles produced using the microfluidic platform of the present invention.

FIG. 17 is a schematic view of a microfluidic platform for producing lipid nanoparticles used in another embodiment of the present invention.

FIG. 18 shows the particle size of lipid nanoparticles produced by a microfluidic chip including an inverted microfluidic channel formed with a serpentine mixer pattern.

FIG. 19 shows the particle size of lipid nanoparticles produced by a microfluidic chip including an inverted microfluidic channel formed with a toroidal mixer pattern.

FIG. 20 is a schematic view of a microfluidic platform for producing lipid nanoparticles used in yet another embodiment of the present invention.

FIG. 21 is a photograph of the microfluidic platform for producing lipid nanoparticles of FIG. 20.

FIG. 22 shows the particle size and encapsulation efficiency of lipid nanoparticles produced by a microfluidic chip including a microfluidic channel formed with a serpentine mixer pattern using a 3D printer.

### DETAILED DESCRIPTION

Specific details of other embodiments are included in the following detailed description and the accompanying drawings. The advantages and features of the present invention, and methods for achieving them, will become apparent from the embodiments described in detail below with reference to the drawings. However, the present invention is not limited to the embodiments disclosed herein, but may be embodied in various different forms. Unless otherwise specified in the following description, all numbers, values, and/or expressions representing components, reaction conditions, and contents of components of the present invention should be understood as being modified in all instances by the term “about,” as those numbers reflect inherent approximations resulting from measurement uncertainties and variations in obtaining such values.

Further, when numerical ranges are disclosed herein, such ranges are continuous, and include all values from the minimum to the maximum of the range unless otherwise indicated. In addition, where the range refers to integers, all integers including and between the minimum and maximum values are encompassed, unless otherwise specified. Moreover, when a range is described in connection with a variable, it should be understood that the variable encompasses all values within the disclosed range including the end points. For example, a range of “5 to 10” encompasses not only the values 5, 6, 7, 8, 9, and 10, but also any sub-ranges such as 6 to 10, 7 to 10, 6 to 9, and 7 to 9, as well as any values between integers that are valid within the scope of the disclosed range, such as 5.5, 6.5, 7.5, 5.5 to 8.5, and 6.5 to 9. Likewise, a range of “10% to 30%” encompasses not only the values 10%, 11%, 12%, 13%, . . . up to 30%, but also any sub-ranges such as 10% to 15%, 12% to 18%, and 20% to 30%, as well as any values between integers that are valid within the scope of the disclosed range, such as 10.5%, 15.5%, and 25.5%.

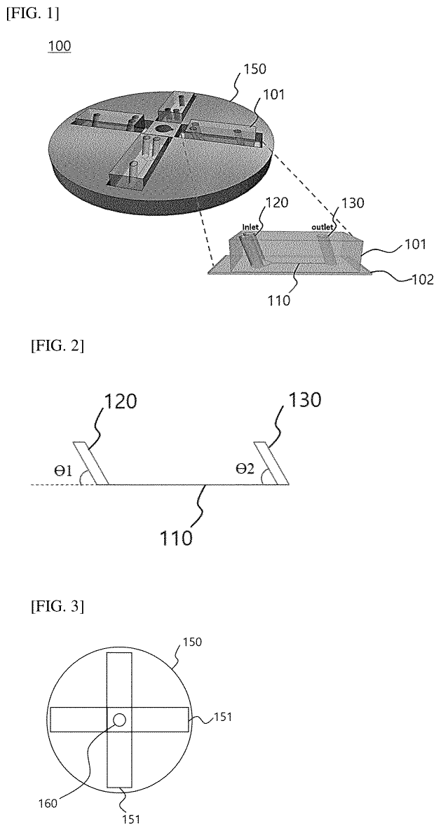

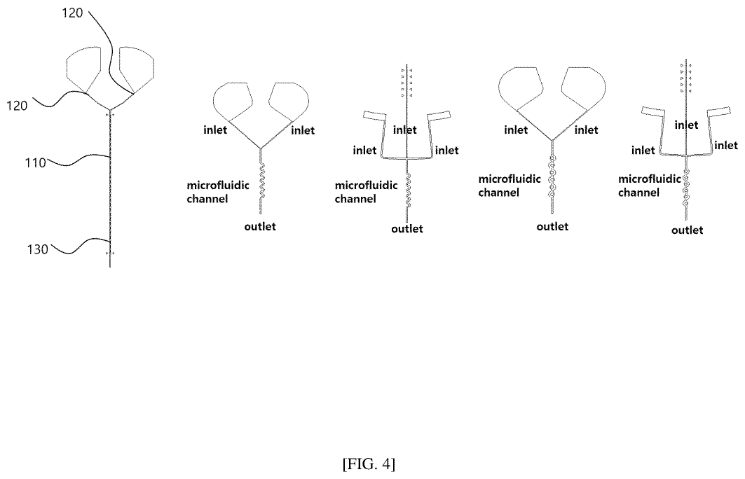

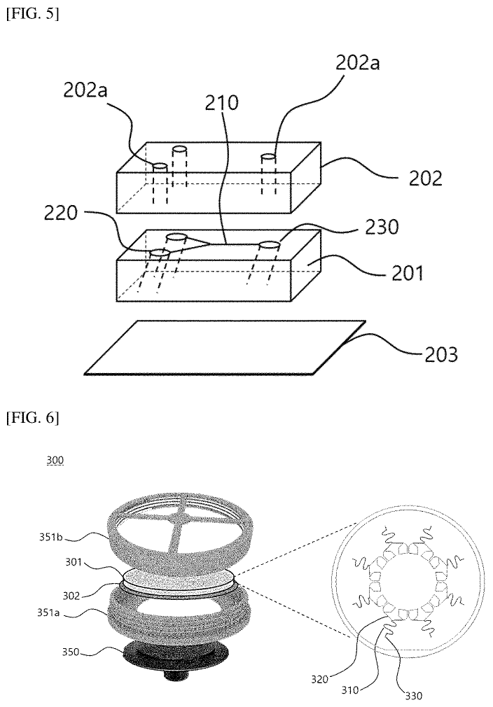

FIG. 1 is a schematic view of a microfluidic platform for producing lipid nanoparticles according to an embodiment of the present invention. FIG. 2 illustrates the angles of the inlet and outlet of the microfluidic chip of FIG. 1. FIG. 3 shows the upper surface of the disk member of FIG. 1. FIG. 4 illustrates a microfluidic channel according to an embodiment of the present invention.

A microfluidic platform (100) for producing lipid nanoparticles according to an embodiment of the present invention includes: a microfluidic channel (110) that utilizes centrifugal force and produces lipid nanoparticles (LNPs) therein; one or more inlets (120) supplying fluid to the microfluidic channel (110); one or more outlets (130) discharging fluid from the microfluidic channel (110); and a disk member (150) rotating the fluid.

Conventional methods for producing lipid nanoparticles, such as pipette mixing or syringe pumps, suffer from drawbacks in that the physical properties of the lipid nanoparticles vary depending on the working environment, device performance, and operator. In addition, although the demand for lipid nanoparticles has increased with the advent of RNA vaccines, there remains a problem that it is difficult to mass-produce lipid nanoparticles with uniform physical properties.

The microfluidic platform (100) according to an embodiment of the present invention can produce lipid nanoparticles uniformly at a small size. The physical properties of the lipid nanoparticles can be controlled uniformly regardless of the operator or working environment, and the lipid nanoparticles can be mass-produced efficiently. The lipid nanoparticles produced using the microfluidic platform of the present invention can be used for various purposes such as intracellular drug delivery, gene therapy, and RNA delivery.

The microfluidic platform (100) includes a microfluidic channel (110) through which the fluid passes while being subjected to centrifugal force, and in which lipid nanoparticles are produced. The fluid is injected into the microfluidic channel (110) through the inlet (120), and the lipid nanoparticles produced in the microfluidic channel (110) are discharged together with the fluid through the outlet (130). The fluid may include a lipid mixture and an active substance mixture for producing lipid nanoparticles. The fluid containing the lipid mixture and the active substance mixture is converted into lipid nanoparticles while passing through the microfluidic channel (110).

The microfluidic channel (110) extends in one direction, and the inlet (120) and the outlet (130) are respectively connected to one end and the other end of the microfluidic channel (110) and extend upward relative to the microfluidic channel (110). The inlet (120) is provided at a first angle (θ1) with respect to a first plane parallel to the ground, and the outlet (130) is provided at a second angle (θ2) with respect to the first plane. For example, when the microfluidic channel (110) is provided on a first plane defined by x- and y-axes, the inlet (120) and the outlet (130) extend in the z-axis direction with first angle (θ1) and second angle (θ2), respectively.

The microfluidic channel (110) is configured to allow the fluid to flow along the first plane and may form a variety of linear or curved flow paths. The inlet (120) and the outlet (130) are formed in an upward direction relative to the microfluidic channel (110), each provided at first angle (θ1) and second angle (θ2), so that the fluid flows into or is discharged from the microfluidic channel (110).

The first angle (θ1) is 20° to 85°, and the second angle (θ2) is not greater than 85°. When the first angle (θ1) and the second angle (θ2) are within the specified ranges, stable lipid nanoparticle size is obtained without significant variation in particle size. Each of the first angle (θ1) and second angle (θ2) is an acute angle measured clockwise or counterclockwise relative to the first plane.

If the first angle (θ1) is less than 20°, excessive shear stress occurs between the inner surface of the inlet (120) and the solvent or solute constituting the fluid, causing problems, and as the spatial footprint of the inlet increases, the chip length becomes longer, making it unsuitable for fabricating small circular chips such as those shown in FIG. 6. If the angle exceeds 85°, the lipid nanoparticles become larger in size and non-uniform. Specifically, the first angle (θ1) may be 20° to 80°, 20° to 75°, 20° to 70°, 30° to 85°, 40° to 85°, 45° to 85°, 45° to 80°, or 45° to 70°.

If the second angle (θ2) exceeds 85°, it becomes difficult to effectively collect lipid nanoparticles. Specifically, the second angle (θ2) may be 80° or less, 75° or less, 70° or less, or 45° to 70°. More specifically, the first angle (θ1) and the second angle (θ2) may be set to the same angle within the range of 45° to 70°. As the first angle and the second angle decrease, the size of the lipid nanoparticles decreases, and within the above ranges, uniform and small lipid nanoparticles can be produced.

The inlet (120) includes two or more inlets through which the lipid mixture and the active substance mixture are introduced into the microfluidic channel (110), and the average velocity of the fluid in the inlet (120) is 1 μL/s to 200 μL/s. By providing two or more inlets (120), the volumetric mixing ratio of the lipid mixture and the active substance mixture constituting the fluid can be controlled more accurately.

If the average velocity of the fluid at the inlet (120) is less than 1 μL/s, mixing of the lipid mixture and the active substance mixture is slowed, resulting in non-uniform mixing, which is undesirable. If the average velocity exceeds 200 μL/s, the particle size cannot be further reduced due to the limiting size of lipid nanoparticles, making the condition unnecessary. Specifically, the average velocity of the fluid at the inlet (120) may be 1.5 μL/s to 200 μL/s, 1.5 μL/s to 150 μL/s, or 1.5 μL/s to 132.5 μL/s.

The inner diameter of the microfluidic channel is 100 μm to 300 μm. If the inner diameter is less than 100 μm, a very high rotational speed is required to achieve the desired flow rate within the microfluidic channel, making stable production of lipid nanoparticles difficult. If the inner diameter exceeds 300 μm, mixing efficiency decreases due to the increased fluid volume, leading to reduced efficiency in producing lipid nanoparticles. Since the cross-section of the microfluidic channel is rectangular, the inner diameter is the hydraulic diameter.

The Reynolds number of the fluid in the microfluidic channel is 5 to 1520. When the Reynolds number is within this range, lipid nanoparticles with uniform physical properties and high encapsulation efficiency can be produced.

The disk member (150) rotates to generate centrifugal force inside the microfluidic channel (110). The rotational speed of the disk member (150) is 500 rpm to 4000 rpm. If the rotational speed is less than 500 rpm, the lipid nanoparticles are too large and non-uniform, which is problematic. If the speed exceeds 4000 rpm, the particle size no longer decreases, and operating beyond 4000 rpm wastes unnecessary energy and lowers process efficiency. Specifically, the rotational speed of the disk member (150) may be 700 rpm to 3500 rpm, 800 rpm to 3000 rpm, 900 rpm to 3000 rpm, or 1000 rpm to 3000 rpm. The average diameter of the lipid nanoparticles is 10 nm to 300 nm. Due to the influence of internal cargo substances, it is difficult to produce nanoparticles smaller than about 10 nm. If the size exceeds 300 nm, intracellular delivery efficiency decreases. The polydispersity index (PDI) is 0.20 or less. Specifically, the average diameter of the lipid nanoparticles may be 10 nm to 250 nm, 10 nm to 200 nm, 20 nm to 300 nm, 40 nm to 300 nm, 60 nm to 300 nm, 80 nm to 300 nm, or 80 nm to 200 nm.

The fluid includes a lipid mixture and an active substance mixture. The lipid mixture includes ionizable lipids, helper lipids, stabilizing lipids, and PEG-lipids. The active substance mixture includes one or more selected from the group consisting of chemotherapeutic agents, small-molecule drugs, proteins, and nucleic acids. The nucleic acid may include one or more selected from the group consisting of genes, recombinant genes, plasmid DNA, DNA molecules, RNA molecules, antisense oligonucleotides, siRNA, rRNA, cDNA, mRNA, shRNA, lncRNA, miRNA, and tRNA.

The ionizable lipid may be a main component that controls the properties of the lipid nanoparticles. The ionizable lipid may include at least one selected from the group consisting of (6Z,9Z,28Z,31Z)-heptatriaconta-6,9,28,31-tetraen-19-yl 4-(dimethylamino)butanoate (D-Lin MC3-DMA or MC3); 9-heptadecanyl 8-{(2-hydroxyethyl) [6-oxo-6-(undecyloxy) hexyl]amino}octanoate (SM-102); [(4-hydroxybutyl) azanediyl]di(hexane-6,1-diyl)bis(2-hexyldecanoate) (ALC-0315); 1,2-distearyloxy-N,N-dimethyl-3-aminopropane (DSDMA); 1,2-dioleyloxy-N,N-dimethyl-3-aminopropane (DODMA); 1,2-dilinoleyloxy-N,N-dimethyl-3-aminopropane (D-Lin-DMA); 2,2-dilinoleyl-4-dimethylaminoethyl-[1,3]-dioxolane (D-Lin-KC2-DMA); N,N-dimethyl-2,3-bis(((9Z,12Z,15Z)-octadeca-9,12,15-trien-1-yl)oxy) propan-1-amine (D-Len-DMA); (Z)—N,N-dimethyl-N—((Z)-octadec-9-en-1-yl) octadec-9-en-1-aminium chloride (DODAC); N,N-dimethyl-N-octadecyloctadecan-1-aminium bromide (DDAB); N-(2-hydroxyethyl)-N,N-dimethyl-2,3-bis(tetradecyloxy) propan-1-aminium bromide (DMRIE); N,N-dimethyl-3,4-dioleyloxybenzylamine (DMOBA); 1,2-N,N′-dioleylcarbamyl-3-dimethylaminopropane (DOcarbDAP); 2,3-dilinoleoyloxy-N,N-dimethylpropylamine (D-Lin-DAP); 1,2-N,N′-dilinoleylcarbamyl-3-dimethylaminopropane (DLincarbDAP); 1,2-dilinoleoylcarbamyl-3-dimethylaminopropane (DLinCDAP); and 2,2-dilinoleyl-4-dimethylaminoethyl-[1,3]-dioxolane (DLin-K-XTC2-DMA).

The helper lipid may be provided to improve the stability of the lipid nanoparticles and to enhance the delivery efficiency of the active substance (e.g., nucleic acids). The helper lipid may include at least one selected from the group consisting of distearoylphosphatidylcholine (DSPC), 1,2-dioleoyl-sn-glycero-3-phosphocholine (DOPC), 1,2-dipalmitoyl-sn-glycero-3-phosphocholine (DPPC), 1,2-dipalmitoyl-sn-glycero-3-phospho-rac-(1-glycerol) (DPPG), 1,2-dioleoyl-sn-glycero-3-phosphoethanolamine (DOPE), 1-palmitoyl-2-oleoyl-sn-glycero-3-phosphocholine (POPC), 1-palmitoyl-2-oleoyl-sn-glycero-3-phosphoethanolamine (POPE), and dioleoyl-sn-glycero-3-phosphoethanolamine-N-(maleimidomethyl) (DOPEmal).

The stabilizer lipid may improve the structural stability of the lipid nanoparticles. The stabilizer lipid may include cholesterol (CHOL).

The PEG-lipid is a PEGylated lipid and may include a PEG derivative bound to a lipid moiety. The PEG-lipid may improve the circulation time of the lipid nanoparticles as nucleic acid carriers and reduce nonspecific uptake. The PEG-lipid may include at least one selected from the group consisting of pegylated diacylglycerol (PEG-DAG), pegylated ceramide lipid (PEG-Cer), pegylated phosphatidylethanolamine lipid (PEG-PE), pegylated succinate diacylglycerol lipid (PEG-S-DAG), pegylated dialkoxypropylcarbamate lipid, 1,2-dimyristoyl-rac-glycero-3-methoxypolyethylene glycol (PEG-DMG), and 1,2-dicapryl-rac-glycero-3-methylpolyoxyethylene glycol (C10 diacylglycerol PEG).

The N/P ratio of the lipid mixture to the active substance mixture may be 1 to 30. When the N/P ratio is within the above range, the encapsulation efficiency and intracellular delivery efficiency of the lipid nanoparticles can be improved. Specifically, the N/P ratio of the active substance mixture may be 1 to 25, 1 to 20, 3 to 25, or 3 to 20.

In the microfluidic platform (100), the microfluidic channel (110), one or more inlets (120), and one or more outlets (130) may be integrally formed on the disk member (150), or may be provided in the form of separate detachable microfluidic chips (101, 102). The microfluidic chip(s) (101, 102) may be provided individually and configured to be reversibly attachable and detachable from the disk member (150).

The microfluidic chip (101, 102) may include a body portion (101) in which the microfluidic channel (110), the inlet (120), and the outlet (130) are formed; and one or more cover portions (102) covering at least one of the first and second surfaces of the body portion (101).

In the body portion (101), the microfluidic channel (110) may be concavely formed inward from one surface of the body portion (101), and the inlet (120) and the outlet (130) may be formed to penetrate through the body portion (101).

Referring to FIG. 3, one surface of the disk member (150) may further include one or more seating portions (151) on which the microfluidic chips (101, 102) are mounted. The seating portion (151) is sized to correspond to the microfluidic chips (101, 102), is concavely formed inward from the surface of the disk member (150), and receives the lower surface of the microfluidic chips (101, 102). The seating portion (151) provided in the disk member (150) allows the microfluidic chips (101, 102) to be stably fixed on the disk member (150) during rotation of the disk member (150), and also guides the positioning of the microfluidic chips (101, 102).

The microfluidic chips (101, 102) may be provided in a box shape and arranged radially with respect to the center portion (160) of the disk member (150). The inlet (120) may be provided adjacent to the center portion (160) of the disk member (150), and the outlet (130) may be provided adjacent to the rim of the disk member (150).

The disk member (150) may rotate through a rotation shaft provided at the center portion (160). By arranging the microfluidic chips (101, 102) radially on the disk member (150), uniform centrifugal force can be applied without variation among the arranged microfluidic chips (101, 102). In addition, by arranging the inlets (120) of the microfluidic chips (101, 102) adjacent to the center portion (160) of the disk member (150), the fluid introduced through the inlets (120) can pass through the microfluidic channel (110) to produce lipid nanoparticles of uniform size.

Referring to FIG. 4, the microfluidic channel (110) may include at least one of a herringbone mixer pattern, a serpentine mixer pattern, and a toroidal mixer pattern. In addition, the microfluidic channel (110) may include two or three inlets (120). The microfluidic channel (110) may be provided in various forms so that the size and properties of the lipid nanoparticles can be controlled to suit different applications.

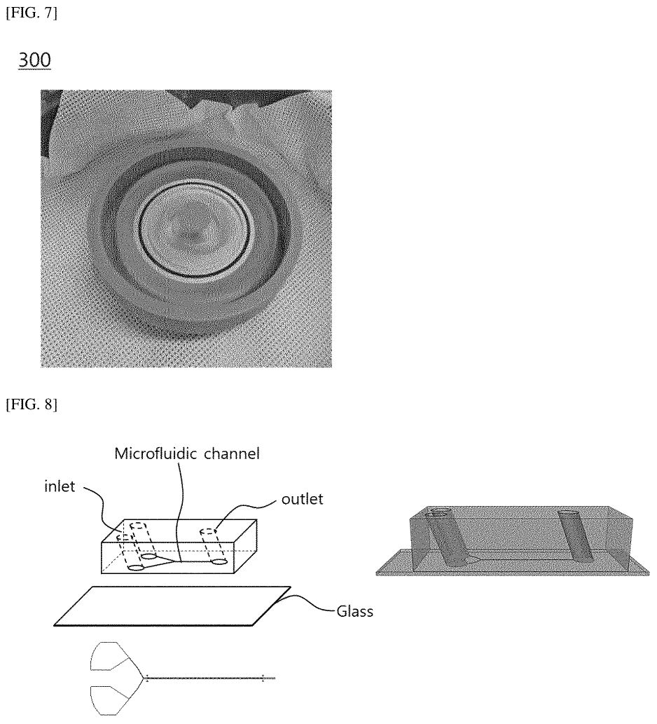

Hereinafter, other embodiments of the present invention will be described with reference to FIGS. 5 to 21. Except for the matters described below, the embodiments are similar to those described with reference to FIGS. 1 to 4, and thus detailed description thereof will be omitted.

FIG. 5 is a schematic view of a microfluidic platform for producing lipid nanoparticles according to another embodiment of the present invention.

The microfluidic chip (201, 202, 203) may include a body portion (201) in which a microfluidic channel (210), an inlet (220), and an outlet (230) are formed; and one or more cover portions (202, 203) covering at least one of the first and second surfaces of the body portion (201). The microfluidic channel (210) may be provided on one surface of the body portion (201) and formed in a concave shape inward, and the inlet (220) and the outlet (230) may be connected to the microfluidic channel (210) and formed to penetrate the other surface of the body portion (201).

The cover portions (202, 203) may include a first cover (202) and a second cover (203). The first cover (202) is configured to cover one surface of the body portion (201) and includes one or more holes (202a) provided at positions corresponding to the inlet (220) and the outlet (230). The second cover (203) is configured to cover the other surface of the body portion (201).

The microfluidic chip (201, 202, 203) according to an embodiment of the present invention may be provided in an inverted form, in which the microfluidic channel (210) is positioned on the upper side, and the inlet (220) and the outlet (230) are oriented toward the lower side.

The fluid may flow into the inlet (220) through the holes (202a) formed in the first cover (202). The holes (202a) provided in the first cover (202) also serve as vent holes, preventing the inlet (220) and the outlet (230) from being sealed and allowing the fluid to flow. When the friction coefficients of the lipid mixture and the active substance mixture constituting the fluid differ, the inflow speeds of the respective substances through the inlet (220) may differ. By adopting the inverted configuration, the microfluidic chip (201, 202, 203) according to an embodiment of the present invention can reduce the influence of the materials constituting the chip on the inflow speed of the substances constituting the fluid.

FIG. 6 is a schematic view of a microfluidic platform for producing lipid nanoparticles according to another embodiment of the present invention. FIG. 7 is a view of the structure of FIG. 6 manufactured by 3D printing.

Referring to FIGS. 6 and 7, the microfluidic platform (300) according to an embodiment of the present invention may be provided as an integral structure having a circular cross-section, rather than in the form of a plurality of separate microfluidic chips (301, 302).

The microfluidic chip (301, 302) may include a body portion (301) in which a microfluidic channel (310), an inlet (320), and an outlet (330) are formed, and a cover portion (302) covering one surface of the body portion (301).

The microfluidic channel (310), the inlet (320), and the outlet (330) provided in the body portion (301) may be configured as a set, and arranged radially with respect to the center of the body portion (301). In this arrangement, the inlet (320) may be provided adjacent to the center of the body portion (301), and the outlet (330) may be provided adjacent to the rim of the body portion (301).

The microfluidic chip (301, 302) may be mounted on and rotated with the disk member (350). The microfluidic chip (301, 302) may be fixed to the disk member (350) by an upper frame (351a) and a lower frame (351b), which secure the microfluidic chip (301, 302) in place. The upper frame (351a) and the lower frame (351b) may be coupled by screw engagement and may accommodate the microfluidic chip (301, 302) therein.

The microfluidic platform (300) according to this embodiment may be manufactured by 3D printing.

According to another aspect of the present invention, the embodiment also provides a method of producing lipid nanoparticles using the above-described microfluidic platform. The method of using the microfluidic platform includes: preparing a lipid mixture and an active substance mixture; injecting the lipid mixture and the active substance mixture into the inlet; and rotating the disk member.

The inlet may include two or more inlets so that the lipid mixture and the active substance mixture can flow into the microfluidic channel. The average velocity of the fluid at the inlet is 1 L/s to 200 μL/s, and the rotational speed of the disk member may be 500 rpm to 4000 rpm. The inlet is connected to one end of the microfluidic channel, and the Reynolds number of the fluid in the microfluidic channel may be 5 to 1520. Specifically, the average velocity of the fluid at the inlet may be 1.5 μL/s to 132.5 μL/s.

The lipid mixture and the active substance mixture are injected into the inlet so that the N/P ratio is between 1 and 30. The lipid mixture and the active substance mixture are mixed under the centrifugal force generated inside the microfluidic channel to produce lipid nanoparticles. The produced lipid nanoparticles are discharged through the outlet connected to the other end of the microfluidic channel.

The fluid may include the lipid mixture and the active substance mixture. The fluid flows through the inlet into the microfluidic channel, where lipid nanoparticles are produced under the centrifugal force.

The lipid nanoparticles thus produced can have uniform size while exhibiting high encapsulation efficiency due to the centrifugal force generated by the rotational speed within the above-described range and the Reynolds number of the fluid in the microfluidic channel. The average diameter of the lipid nanoparticles may be 10 nm to 300 nm.

### EXAMPLES AND COMPARATIVE EXAMPLES

The following describes examples and comparative examples of the present invention. However, the following examples are merely preferred embodiments of the present invention, and the scope of rights of the present invention is not limited thereto.

### 1. Preparation of Lipid Mixture and Nucleic Acid Solution

In order to produce lipid nanoparticles, a lipid mixture and a nucleic acid solution were prepared as follows.

The lipid mixture was prepared using D-Lin-MC3-DMA as an ionizable lipid, distearoylphosphatidylcholine (DSPC) as a helper lipid, cholesterol as a stabilizer lipid, and DMG-PEG 2000 as a PEG-lipid. These components were mixed in ethanol at a molar ratio of D-Lin-MC3-DMA:DSPC:Cholesterol:DMG-PEG 2000=50:10:38.5:1.5 to prepare the lipid mixture.

For the nucleic acid solution, ssDNA was prepared using 10 mM citrate buffer solution (pH 3), and F-Luc-mRNA and eGFP-mRNA were each prepared using 25 mM sodium acetate buffer solution (pH 5).

The lipid mixture was prepared with the same composition as described above, and the nucleic acid solution was prepared as shown in Table 1 below. The lipid mixture and the nucleic acid solution were separately injected into the inlets at a volume ratio of 1:3, and mixing was performed by the rotational force of the disk. Furthermore, the N/P ratio of the lipid mixture and the nucleic acid solution was adjusted and mixed as specified in Table 1.

TABLE 1

| | | | mixing with the lipid mixture | |
|---|---|---|---|---|
| classifi- | Nucleic Acid | | Lipid concentration in | N/P |
| cation | Nucleic acid | Solvent | lipid mixture(mM) | ratio |
| ssDNA | ssDNA | citrate buffer | 6.25 | 6 |
| | | solution | | |
| F-Luc | F-Luc- | sodium acetate | 12.5 | 5.57 |
| | mRNA | buffer solution | | |
| eGFP | eGFP- | sodium acetate | 12.5 | 5.57 |
| | mRNA | buffer solution” | | |

### 2. Production of Lipid Nanoparticles

### (1) Production of Lipid Nanoparticles Using the Microfluidic Platform

FIG. 8 is a schematic diagram of a microfluidic chip used in the microfluidic platform for producing lipid nanoparticles according to an embodiment. As shown in FIG. 8, the microfluidic chip was fabricated by covering a box-shaped PDMS (polydimethylsiloxane) structure, in which a microfluidic channel, inlet, and outlet were formed, with a glass plate. The microfluidic channel was designed using AutoCAD and fabricated with a herringbone mixer pattern by photolithography using SU-8 photoresist (MicroChem). The microfluidic channel was then formed in the PDMS substrate using soft lithography. The inlet and outlet were created by punching holes with a punch tool. The inlets and outlets were fabricated to have the same angle in parallel, and chips were produced and evaluated at tilt angles of 70° and 90°. The fabricated microfluidic chip was mounted on a disk member, as shown in FIG. 1, and used for the production of lipid nanoparticles. The disk member was fabricated using a 3D printer.

The previously prepared lipid mixture and nucleic acid solution were separately injected into the inlets of the microfluidic chip. Each microfluidic chip, into which the lipid mixture and nucleic acid solution were introduced, was mounted on the disk member, and the disk member was rotated. After rotation, the lipid nanoparticles produced were collected through the outlet.

### (2) Production of Lipid Nanoparticles by Pipette Mixing

For comparison, lipid nanoparticles were also produced using the same lipid mixture and nucleic acid solution at the same ratio by employing a conventional pipette mixing method. In this procedure, pipette mixing was performed by aspirating and dispensing 60 times to ensure uniform mixing of the lipid mixture and nucleic acid solution, followed by standing at room temperature for 10 minutes after mixing.

### (3) Production of Lipid Nanoparticles by Syringe Pump Mixing

Syringe pump mixing was carried out using the following setup. A syringe infusion pump was connected to syringes and used to pump at a controlled flow rate. The lipid solution and nucleic acid solution were introduced at a 1:3 flow ratio into a PDMS chip containing a herringbone microfluidic mixer, and mixing was performed. The syringes and inlets were connected with peek tubing, and the outlet was also connected with peek tubing to collect the discharged mixture. Syringe pump mixing is a technique similar to that of the disk platform; however, the fluid flow is controlled by the syringe pump. The herringbone microfluidic mixer was fabricated with reference to prior art (Abraham D. Stroock, “Chaotic Mixer for Microchannels,” Science, 295 (5555): 647-651, 2002).

The same lipid mixture and nucleic acid solution as described in the Example were each loaded into 1 mL syringes. The prepared syringes were connected to the microfluidic chip using a conventional syringe pump and peek tubing to inject the solutions and perform mixing. After mixing, the mixture was allowed to stand at room temperature for 10 minutes.

### 3. Measurement of Lipid Nanoparticles

The size of the lipid nanoparticles (LNPs) was measured using a dynamic light scattering analyzer (DLS; Zetasizer Nano S90, Malvern). A sample was prepared by diluting 10 μL of the lipid nanoparticles in 990 μL of PBS, and the measurement was carried out using this sample.

The encapsulation efficiency of the lipid nanoparticles, which refers to the gene entrapment efficiency, was quantified using the Quant-iT RiboGreen assay (Thermo Fisher).

### 4. Evaluation According to Inlet and Outlet Angles

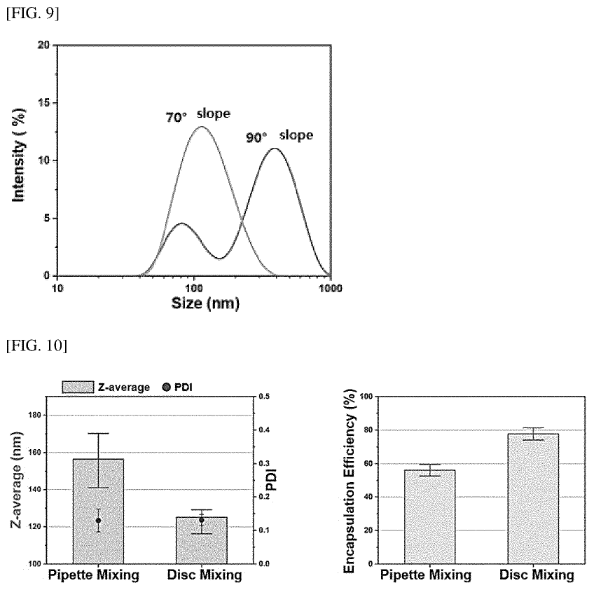

FIG. 9 shows the particle sizes of lipid nanoparticles produced using microfluidic chips with different inlet angles according to an embodiment of the present invention.

In FIG. 9, the inlets of the microfluidic chip were fabricated with tilt angles of 70° and 90° (θ, see FIG. 3), and the outlets were fabricated to have the same angles as the inlets. Table 2 shows the particle size and polydispersity index (PDI) of lipid nanoparticles produced depending on the inlet angle.

TABLE 2

| | | Disk | | | |
|---|---|---|---|---|---|
| | Inlet | Rotation | Nucleic | Average | |
| Manufacturing | Angle | Speed | Acid | Particle | |
| Method | (°) | (rpm) | Solution | Size (nm) | PDI |
| Microfluidic | 70 | 1600 | ssDNA | 118.2 | 0.195 |
| platform | 70 | 1600 | ssDNA | 242.2 | 0.452 |

Referring to FIG. 9 and Table 2, the size and PDI of the lipid nanoparticles produced varied depending on the inclination angle of the inlet. When the inlet angle decreased from 90° to 70°, the lipid nanoparticles exhibited a more uniform particle size, and the PDI was reduced.

### 4. Lipid Nanoparticles Produced by Microfluidic Platform, Pipette Mixing, and Syringe Pump Mixing

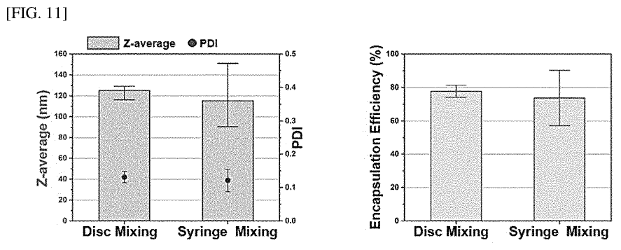

FIG. 10 shows a comparison of lipid nanoparticles produced using the microfluidic platform according to an embodiment of the present invention and those produced by pipette mixing in a comparative example. FIG. 11 shows a comparison of lipid nanoparticles produced using the microfluidic platform according to an embodiment of the present invention and those produced by syringe pump mixing in a comparative example.

Table 3 presents the particle size, PDI, and encapsulation efficiency of lipid nanoparticles according to FIG. 10, and Table 4 presents the particle size, PDI, and encapsulation efficiency of lipid nanoparticles according to FIG. 11. In FIGS. 10 and 11, the inlet angle in the microfluidic platform was 70°, and the rotational speed of the disk member was 1600 rpm.

TABLE 3

| Manufacturing | Nucleic Acid | Z-average | | Encapsulation |
|---|---|---|---|---|
| Method | Solution | (nm) | PDI | efficiency (%) |
| Microfluidic | ssDNA | 125.1 | 0.131 | 77.7 |
| platform (disc | | | | |
| mixing) | | | | |
| Pipette mixing | ssDNA | 156.4 | 0.130 | 56.1 |

TABLE 4

| Manufacturing | Nucleic Acid | Z-average | | Encapsulation |
|---|---|---|---|---|
| Method | Solution | (nm) | PDI | efficiency (%) |
| Microfluidic | ssDNA | 125.1 | 0.131 | 77.7 |
| platform (disc | | | | |
| mixing) | | | | |
| Syring mixing | ssDNA | 115.3 | 0.121 | 73.7 |

Referring to FIG. 10 and Table 2, lipid nanoparticles produced according to an embodiment of the present invention exhibited an average particle size of approximately 125.1 nm with a PDI of about 0.131, whereas lipid nanoparticles produced by conventional pipette mixing exhibited an average particle size of approximately 156.4 nm. Despite the larger size, the PDI of the nanoparticles prepared by pipette mixing was similar to that of the nanoparticles produced according to the present invention. Since the PDI is associated with particle size, even if the distribution is the same, the PDI value becomes smaller as the particle size increases. Therefore, when the PDI values are the same, the smaller lipid nanoparticles produced according to the present invention are confirmed to be more uniform. Furthermore, in terms of encapsulation efficiency, the lipid nanoparticles produced according to the present invention were superior to those produced by pipette mixing.

Referring to FIG. 11 and Table 3, lipid nanoparticles produced according to an embodiment of the present invention and those produced by syringe pump mixing exhibited similar average particle sizes and PDIs. However, the encapsulation efficiency of the lipid nanoparticles produced according to the present invention was higher than that of those produced by syringe pump mixing.

Accordingly, compared with conventional pipette mixing and syringe pump mixing methods, lipid nanoparticles produced according to the present invention were smaller in average particle size, more uniform, and exhibited superior encapsulation efficiency.

### 5. Evaluation of Intracellular Delivery Efficiency According to LNP Production Method

The intracellular delivery efficiency of lipid nanoparticles prepared using the microfluidic platform of the present invention and those prepared by pipette mixing was evaluated using HEK293T cells.

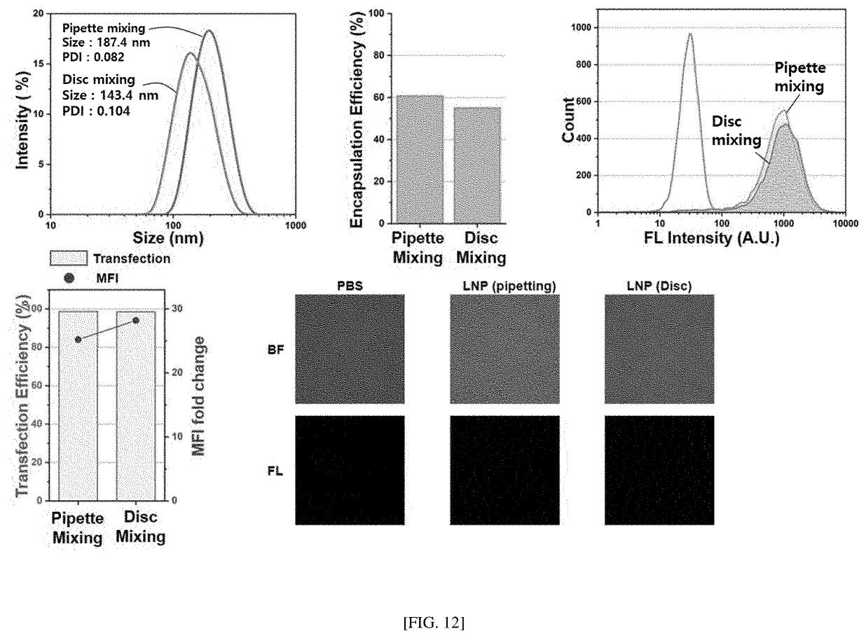

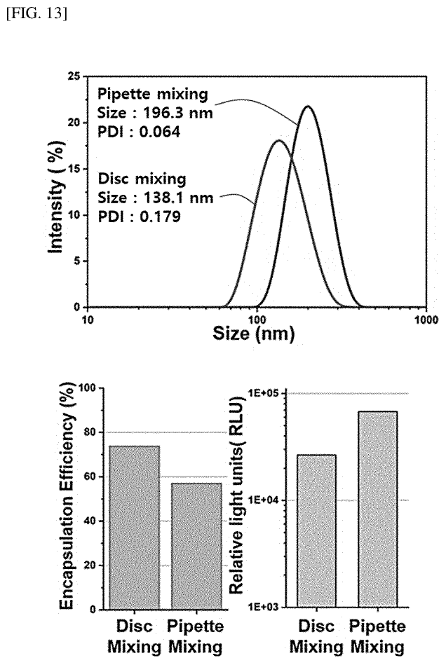

FIG. 12 shows the intracellular delivery efficiency of lipid nanoparticles containing eGFP-mRNA produced by different methods. FIG. 13 shows the intracellular delivery efficiency of lipid nanoparticles containing F-Luc-mRNA produced by different methods. In the microfluidic platform of FIGS. 12 and 13, the inlet angle was set at 70°, and the rotational speed of the disk member was 1600 rpm.

Table 5 presents the comparative results of intracellular delivery efficiency according to FIGS. 12 and 13.

TABLE 5

| | | Nucleic | Z- | | Encapsulation | transfection | | Relative |
|---|---|---|---|---|---|---|---|---|
| Manufacturing | | acid | average | | efficiency | efficiency | | light |
| Method | cell | solution | (nm) | PDI | (%) | (%) | MFI | unit |
| Microfluidic | HEK293 | eGFP | 143.4 | 0.104 | 60.8 | 98.45 | 28.2 | — |
| platform (disc | | | | | | | | |
| mixing) | | | | | | | | |
| Pipette mixing | HEK293 | eGFP | 187.4 | 0.082 | 55.1 | 98.64 | 25.2 | — |
| Microfluidic | HEK293 | F-Luc | 138.1 | 0.179 | 73.7 | — | — | 67719 |
| platform (disc | | | | | | | | |
| mixing) | | | | | | | | |
| Pipette mixing | HEK293 | F-Luc | 196.3 | 0.064 | 57.0 | — | — | 26563 |

Referring to FIG. 12 and Table 5, when lipid nanoparticles containing eGFP-mRNA were produced by the microfluidic platform and by pipette mixing, the particle size and PDI were similar in both cases. However, the encapsulation efficiency of the lipid nanoparticles produced using the microfluidic platform of the present invention was superior. In addition, the intracellular delivery (transfection) efficiency of lipid nanoparticles produced by the microfluidic platform and by pipette mixing was found to be similar.

Referring to FIG. 13 and Table 5, in the case of lipid nanoparticles containing F-Luc-mRNA, the particle size and PDI were smaller and more uniform when produced using the microfluidic platform compared to pipette mixing. Moreover, the lipid nanoparticles produced by the microfluidic platform exhibited higher encapsulation efficiency than those produced by pipette mixing; however, their intracellular delivery efficiency was slightly lower compared to pipette mixing.

### 6. Evaluation of Intracellular Delivery Efficiency According to Cell Type

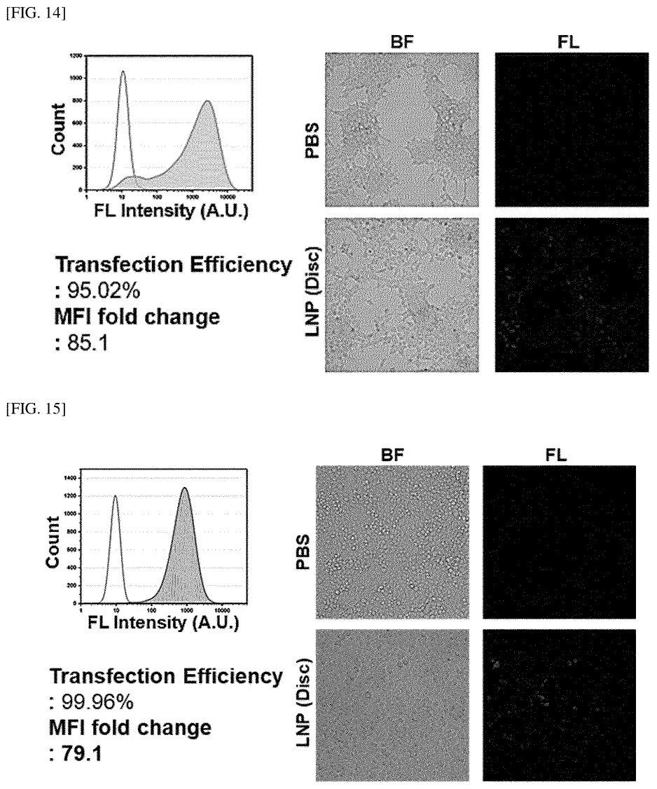

FIG. 14 shows the delivery efficiency of lipid nanoparticles produced using the microfluidic platform of the present invention in HEK293 cells.

FIG. 15 shows the delivery efficiency of lipid nanoparticles produced using the microfluidic platform of the present invention in CHO-K1 cells.

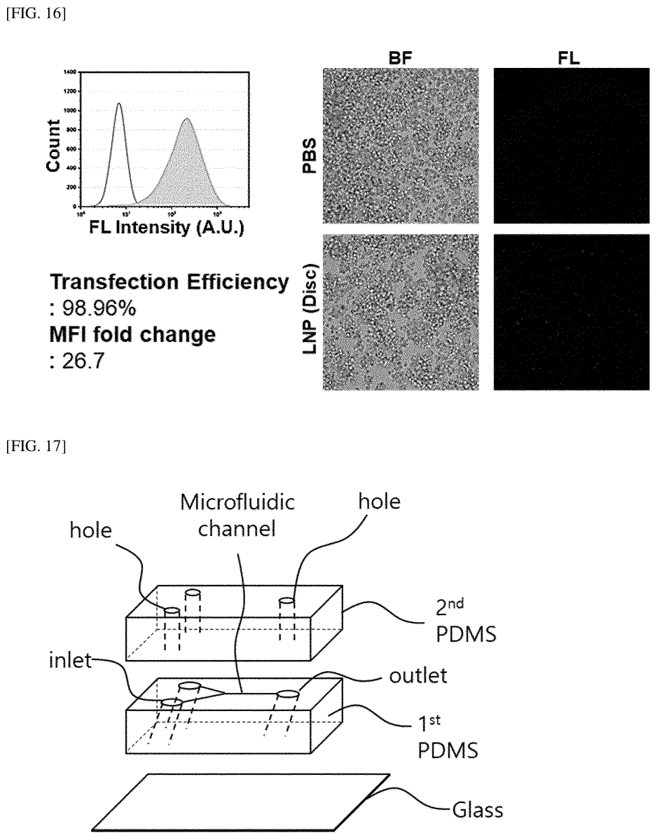

FIG. 16 shows the delivery efficiency of lipid nanoparticles produced using the microfluidic platform of the present invention in Jurkat cells.

In FIGS. 14 to 15, the inlet angle was 70°, and the rotational speed of the disk member was 2800 rpm.

Table 6 presents the results of intracellular delivery efficiency of lipid nanoparticles produced using the microfluidic platform of the present invention in various cell types.

TABLE 6

| | | Nucleic | Z- | | Encapsulation | transfection | |
|---|---|---|---|---|---|---|---|
| Manufacturing | | acid | average | | efficiency | efficiency | |
| Method | cell | solution | (nm) | PDI | (%) | (%) | MFI |
| Microfluidic | HEK293 | eGFP | 104.9 | 0.179 | 84.2% | 95.02 | 85.1 |
| platform (disc | | | | | | | |
| mixing) | | | | | | | |
| Microfluidic | CHO-K1 | eGFP | 104.9 | 0.179 | 84.2% | 99.96 | 79.1 |
| platform (disc | | | | | | | |
| mixing) | | | | | | | |
| Microfluidic | Jurkat | eGFP | 104.9 | 0.179 | 84.2% | 98.96 | 26.7 |
| platform (disc | | | | | | | |
| mixing) | | | | | | | |

Referring to FIGS. 14 to 15 and Table 6, lipid nanoparticles were produced using the microfluidic platform of the present invention, and the delivery efficiency of the produced lipid nanoparticles was evaluated across different cell types.

In FIG. 14, HEK293 cells were seeded in 12-well plates at a density of 4×10⁵ cells/well. After 24 h, lipid nanoparticles (LNPs) containing eGFP mRNA were diluted in serum-free medium to an eGFP mRNA concentration of 500 ng/mL. The existing culture medium was then replaced with the LNP-containing medium to treat the cells. After an additional 24 h, the intracellular delivery of eGFP mRNA was confirmed by fluorescence microscopy and flow cytometry. Referring to FIG. 14 and Table 6, lipid nanoparticles produced according to the present invention exhibited a high delivery efficiency of 95.02% in HEK293 cells.

In FIG. 15, CHO-K1 cells were seeded in 12-well plates at a density of 2×10⁵ cells/well and cultured for 24 h. LNPs containing eGFP mRNA were then diluted in serum-free medium to an eGFP mRNA concentration of 500 ng/mL, and the existing medium was replaced with the LNP-containing medium. After 24 h, the intracellular delivery of eGFP mRNA was analyzed by fluorescence microscopy and flow cytometry. Referring to FIG. 15 and Table 6, lipid nanoparticles produced according to the present invention exhibited a high delivery efficiency of 99.96% in CHO-K1 cells.

In FIG. 16, Jurkat cells were seeded in 24-well plates at a density of 1×10⁵ cells/well. After 24 h, LNPs containing eGFP mRNA were diluted in serum-free medium to an eGFP mRNA concentration of 500 ng/mL, and the existing medium was replaced with the LNP-containing medium. After another 24 h, the intracellular delivery of eGFP mRNA was analyzed by fluorescence microscopy and flow cytometry. Referring to FIG. 16 and Table 6, lipid nanoparticles produced according to the present invention exhibited a high delivery efficiency of 98.96% in Jurkat cells.

Accordingly, lipid nanoparticles produced using the microfluidic platform of the present invention demonstrated high intracellular delivery efficiency regardless of cell type.

### 7. Verification of Lipid Nanoparticle Size According to Microfluidic Chip Configuration

### (1) Preparation of Lipid Mixture and Nucleic Acid Solution

To produce lipid nanoparticles, a lipid mixture and a nucleic acid solution were prepared as follows. The lipid mixture was prepared using D-Lin-MC3-DMA as an ionizable lipid, distearoylphosphatidylcholine (DSPC) as a helper lipid, cholesterol as a stabilizer lipid, and DMG-PEG 2000 as a PEG-lipid. These components were mixed in ethanol at a molar ratio of D-Lin-MC3-DMA:DSPC:Cholesterol:DMG-PEG 2000=50:10:38.5:1.5. The nucleic acid solution was prepared by dissolving ssDNA in 10 mM citrate buffer solution (pH 3). A nucleic acid solution with a lipid concentration of 6.25 mM and an N/P ratio of 6 was prepared, and the lipid mixture and nucleic acid solution were mixed at a volume ratio of 1:3.

### (2) Production of Lipid Nanoparticles Using Microfluidic Platform

FIG. 17 is a schematic diagram of a microfluidic platform for producing lipid nanoparticles according to another embodiment of the present invention. FIG. 17 illustrates a structure similar to the microfluidic platform shown in FIG. 8, except that the microfluidic channel was fabricated so as not to be in direct contact with the glass substrate. In this configuration, the microfluidic channel was positioned on the upper side of the microfluidic chip, such that the channel was inverted compared to the structure of FIG. 8.

As shown in FIG. 17, the microfluidic chip was fabricated by forming a box-shaped first PDMS (polydimethylsiloxane) substrate containing the microfluidic channel, inlet, and outlet, and then fabricating a second PDMS substrate with holes formed therein. The microfluidic channel was designed using AutoCAD and fabricated by photolithography with SU-8 photoresist (MicroChem), in the form of either a serpentine mixer pattern or a toroidal mixer pattern. The channel was formed in the first PDMS substrate using soft lithography. The inlet and outlet were created by punching holes, which were fabricated to be parallel at the same angle, specifically at a tilt angle of 70°. In addition, three holes were formed in the second PDMS by hole punching, positioned to correspond to the structure of the first PDMS.

The microfluidic chip was assembled by placing the second PDMS over the upper side of the first PDMS (where the microfluidic channel was located) and providing a glass plate under the lower side of the first PDMS. The resulting microfluidic chip was used with a disk member, as shown in FIG. 1, to produce lipid nanoparticles. The disk member was fabricated using a 3D printer.

The previously prepared lipid mixture and nucleic acid solution were injected into the inlets of the microfluidic chip. After injection, the microfluidic chip was mounted on the disk member, and the disk member was rotated at 2200 rpm. After rotation, the lipid nanoparticles produced were collected through the outlet.

### (3) Evaluation According to Microfluidic Channel Pattern and Number of Inlets

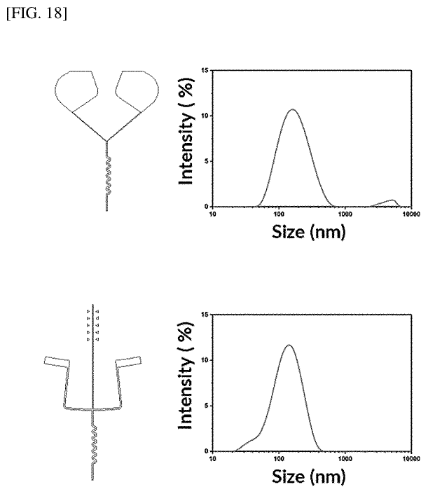

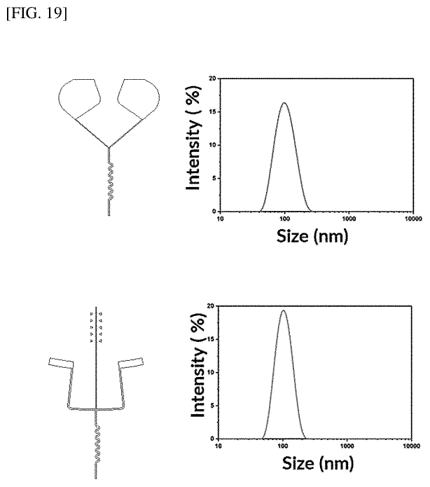

FIG. 18 shows the particle size of lipid nanoparticles produced using a microfluidic chip having an inverted microfluidic channel formed with a serpentine mixer pattern. FIG. 19 shows the particle size of lipid nanoparticles produced using a microfluidic chip having an inverted microfluidic channel formed with a toroidal mixer pattern.

Table 7 presents the particle size of lipid nanoparticles depending on the type of microfluidic channel pattern and the number of inlets.

TABLE 7

| | | | | | average | |
|---|---|---|---|---|---|---|
| | Rotational | | | | particle size | |
| | Speed of | Microfluidic | | nucleic | of the lipid | |
| Manufacturing | the Disc | Channel | | acid | nanoparticles | |
| Method | Member (rpm) | Pattern | Inlet | solution | (nm) | PDI |
| Microfluidic | 2200 | serpentine | two inlets | ssDNA | 155.6 | 0.233 |
| platform | | mixer | | | | |
| (inverted | 2200 | serpentine | three inlets | ssDNA | 119.5 | 0.246 |
| microfluidic | | mixer | | | | |
| channel) | 2200 | toroidal mixer | two inlets | ssDNA | 97.05 | 0.100 |
| | 2200 | toroidal mixer | three inlets | ssDNA | 97.05 | 0.078 |

In this embodiment, a microfluidic chip having an inverted microfluidic channel was employed in order to examine differences relative to a non-inverted microfluidic channel. In the case of the previously tested non-inverted microfluidic channel, it was observed that, prior to rotation of the disc member, the lipid mixture was preferentially introduced into the microfluidic channel due to ethanol used as the solvent of the lipid mixture. To reduce this effect, lipid nanoparticles were prepared using a microfluidic channel fabricated in an inverted configuration.

Referring to FIG. 18, it was found that the average particle size of the lipid nanoparticles was substantially the same regardless of whether two inlets or three inlets were employed. Moreover, as shown in FIG. 19, the number of inlets likewise had little effect on the average particle size of the lipid nanoparticles in a different microfluidic channel pattern. As further illustrated in FIGS. 18 and 19, the toroidal mixer pattern of the microfluidic channel produced smaller lipid nanoparticles compared to the serpentine mixer pattern. This is believed to result from differences in fluid velocity within the microfluidic channel depending on the pattern configuration.

### 8. Verification of Lipid Nanoparticle Size According to Microfluidic Chip Fabrication Method

### (1) Preparation of Lipid Mixture and Nucleic Acid Solution

To manufacture lipid nanoparticles (LNPs), a lipid mixture and a nucleic acid solution were separately prepared as follows.

The lipid mixture was prepared by using D-Lin-MC3-DMA as an ionizable lipid, distearoylphosphatidylcholine (DSPC) as a helper lipid, cholesterol as a stabilizer lipid, and DMG-PEG 2000 as a PEG-lipid. These components were mixed in ethanol at a molar ratio of D-Lin-MC3-DMA:DSPC:Cholesterol:DMG-PEG 2000=50:10:38.5:1.5 to prepare the lipid mixture.

The nucleic acid solution was prepared by dissolving ssDNA in a 10 mM citrate buffer solution (pH 3). The nucleic acid solution was adjusted to an N/P ratio of 6 with a 6.25 mM lipid mixture, and the lipid mixture and nucleic acid solution were combined at a volume ratio of 1:5.

### (2) LNP Fabrication Using the Microfluidic Platform

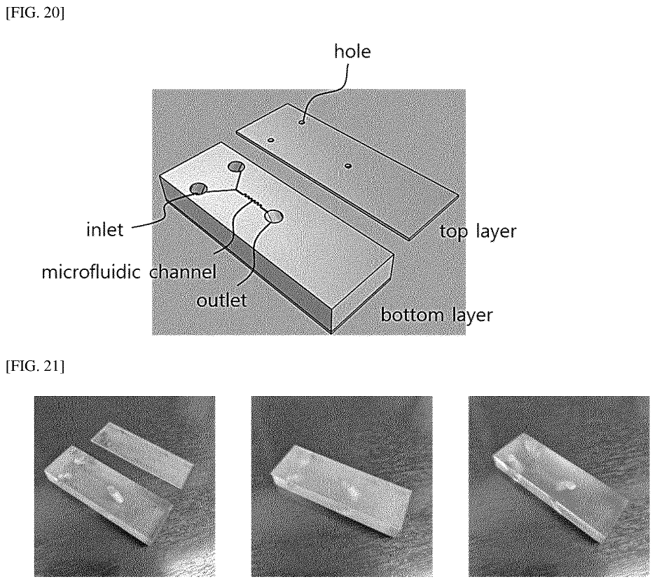

FIG. 20 illustrates a schematic of another embodiment of the microfluidic platform for manufacturing lipid nanoparticles according to the present invention. FIG. 21 shows images of the microfluidic platform of FIG. 20, including the top layer and the bottom layer, the assembled configuration with the top layer placed over the bottom layer, and the bottom surface of the bottom layer. The microfluidic chip shown in FIG. 21 was fabricated in an inverted configuration.

In contrast to the previously described microfluidic platforms, the microfluidic chip of FIGS. 20 and 21 was fabricated not by photolithography but by three-dimensional (3D) printing. The microfluidic channel was designed in a serpentine mixer pattern and manufactured using a PC-like resin (V-FT resin) by stereolithography (SLA).

During use, the bottom layer (reservoir) was loaded with the lipid mixture and nucleic acid solution in the desired ratio, and a top layer with vent holes was placed and sealed over the bottom layer. The assembled microfluidic chip was then mounted on the disc member. Thereafter, the disc member was rotated at various speeds, and after rotation ceased, the lipid nanoparticles formed within the microfluidic channel were collected through the outlet.

### (3) Evaluation According to Microfluidic Chip Fabrication Method and Disc Member Rotation Speed

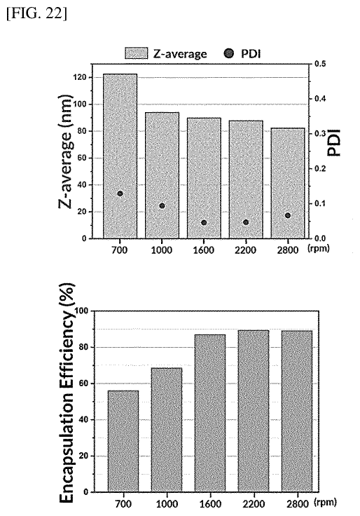

FIG. 22 illustrates the particle size and encapsulation efficiency of lipid nanoparticles prepared using a microfluidic chip fabricated by 3D printing, wherein the chip included a microfluidic channel in a serpentine mixer pattern.

Table 8 summarizes the particle size and encapsulation efficiency of lipid nanoparticles prepared using the 3D-printed microfluidic chip, evaluated at different rotation speeds of the disc member.

TABLE 8

| | | | | average | | |
|---|---|---|---|---|---|---|
| | Rotational | | | of the lipid | | |
| | Speed of | Microfluidic | nucleic | particle size | | Encapsulation |
| Manufacturing | the Disc | Channel | acid | nanoparticles | | efficiency |
| Method | Member (rpm) | Pattern | solution | (nm) | PDI | (%) |
| Microfluidic | 700 | serpentine | ssDNA | 122.5 | 0.129 | 56 |
| platform | | mixer | | | | |
| (3D | 1000 | serpentine | ssDNA | 103.6 | 0.114 | 60.2 |
| printing) | | mixer | | | | |
| | 1600 | serpentine | ssDNA | 99.10 | 0.024 | 83.3 |
| | | mixer | | | | |
| | 2200 | serpentine | ssDNA | 94.84 | 0.072 | 90.8 |
| | | mixer | | | | |
| | 2800 | serpentine | ssDNA | 93.24 | 0.066 | 87 |
| | | mixer | | | | |

Referring to FIG. 21 and Table 8, it was confirmed that lipid nanoparticles could also be uniformly produced in small sizes using the microfluidic chip fabricated by 3D printing. Furthermore, the lipid nanoparticles thus prepared exhibited high encapsulation efficiency.

When examining the effect of the rotation speed of the disc member, it was observed that as the rotation speed increased, the particle size of the lipid nanoparticles decreased, while the encapsulation efficiency increased. Beyond approximately 1600 rpm, the particle size and encapsulation efficiency of the lipid nanoparticles were found to remain substantially similar. Accordingly, it was confirmed that the microfluidic platform according to the embodiments of the present invention is capable of uniformly controlling the lipid nanoparticles to a smaller size.

It will be understood by those skilled in the art to which the present invention pertains that the invention may be embodied in other specific forms without departing from the spirit or essential characteristics thereof. Accordingly, the above-described embodiments are to be considered in all respects as illustrative and not restrictive. The scope of the invention is defined by the appended claims rather than by the foregoing detailed description, and all changes and modifications derived from the meaning and scope of the claims and their equivalents are to be construed as being included within the scope of the present invention.

1. A microfluidic platform for producing lipid nanoparticles (LNPs), comprising:
a microfluidic channel configured to produce lipid nanoparticles using centrifugal force;
at least one inlet configured to supply a fluid to the microfluidic channel;
at least one outlet configured to discharge the fluid from the microfluidic channel; and
a disk member configured to rotate the fluid.

2. The microfluidic platform of claim 1,
wherein the microfluidic channel extends in one direction,
the inlet and the outlet are respectively connected to one end and the other end of the microfluidic channel and extend upward relative to the microfluidic channel,
the inlet is provided at a first angle with respect to a first plane parallel to the ground, and the outlet is provided at a second angle with respect to the first plane.

3. The microfluidic platform of claim 2,
wherein the first angle is 20° to 85°,
the second angle is not greater than 85°,
and the size of the lipid nanoparticles decreases as the first and second angles decrease.

4. The microfluidic platform of claim 3,
wherein the first angle and the second angle are identical and are in a range of 45° to 70°.

5. The microfluidic platform of claim 1,
wherein the microfluidic channel has an inner diameter of 100 μm to 300 μm,
and a Reynolds number of the fluid in the microfluidic channel is 5 to 1520.

6. The microfluidic platform of claim 1,
wherein the disk member is rotated to generate the centrifugal force inside the microfluidic channel,
the disk member has a rotational speed of 500 rpm to 4000 rpm,
the lipid nanoparticles have an average diameter of 10 nm to 300 nm, and
a polydispersity index (PDI) of 0.20 or less.

7. The microfluidic platform of claim 6,
wherein the inlet comprises two or more inlets through which a lipid mixture and an active substance mixture are introduced into the microfluidic channel,
and an average flow rate of fluid at the inlet is 1 μL/s to 200 L/s.

8. The microfluidic platform of claim 7,
wherein the lipid mixture comprises an ionizable lipid, a helper lipid, a stabilizer lipid, and a PEG-lipid,
the active substance mixture comprises at least one selected from the group consisting of a chemotherapeutic agent, a small molecule drug, a protein, and a nucleic acid, and
an N/P ratio of the lipid mixture to the active substance mixture is 1 to 30.

9. The microfluidic platform of claim 1, further comprising at least one microfluidic chip including the microfluidic channel, the at least one inlet, and the at least one outlet,
wherein the at least one microfluidic chip is detachably mountable on one surface of the disk member.

10. The microfluidic platform of claim 9,
wherein the microfluidic chip comprises:
a body portion in which the microfluidic channel, the inlet, and the outlet are formed; and
at least one cover portion covering one or both surfaces of the body portion,
wherein the microfluidic channel is recessed inward from one surface of the body portion, and
the inlet and the outlet are formed to penetrate through the body portion.

11. The microfluidic platform of claim 10,
wherein the cover portion comprises a first cover and a second cover,
the first cover covers one surface of the body portion and comprises at least one hole positioned corresponding to the inlet and the outlet, and
the second cover covers the other surface of the body portion.

12. The microfluidic platform of claim 9,
wherein one surface of the disk member further comprises at least one seating portion for seating the microfluidic chip,
the seating portion being recessed inward from the one surface of the disk member and sized to correspond to the microfluidic chip so that a lower surface of the microfluidic chip is inserted therein.

13. The microfluidic platform of claim 9,
wherein the microfluidic chip is box-shaped and arranged radially with respect to a central portion of the disk member,
the inlet is disposed adjacent to the central portion of the disk member, and
the outlet is disposed adjacent to an edge of the disk member.

14. The microfluidic platform of claim 1,
wherein the microfluidic channel comprises at least one selected from the group consisting of a herringbone mixer pattern, a serpentine mixer pattern, and a toroidal mixer pattern.

15. A method of producing lipid nanoparticles using the microfluidic platform according to claim 1, comprising:
preparing a lipid mixture and an active substance mixture;
injecting the lipid mixture and the active substance mixture into the inlet; and
rotating the disk member.

16. The method of claim 15, wherein the inlet comprises two or more inlets through which the lipid mixture and the active substance mixture are introduced into the microfluidic channel,
an average flow rate of fluid at the inlet is 1 μL/s to 200 μL/s, and
the rotational speed of the disk member is 500 rpm to 4000 rpm.

17. The method of claim 15,
wherein the inlet is connected to one end of the microfluidic channel,
and a Reynolds number of the fluid inside the microfluidic channel is 5 to 1520.

18. The method of claim 15,
wherein the lipid mixture and the active substance mixture are injected into the inlet to achieve an N/P ratio of 1 to 30,
the lipid mixture and the active substance mixture are mixed by centrifugal force generated inside the microfluidic channel to produce lipid nanoparticles, and
the lipid nanoparticles are discharged through the outlet connected to the other end of the microfluidic channel,
wherein the lipid nanoparticles have an average diameter of 10 nm to 300 nm.
